(19) United States
(12) Patent Application Publication
(10) Pub. No.: US 2019/0231576 A1
(43) Pub. Date: Aug. 1, 2019

(54) Body Tension Band

(71) Applicant: Joseph Lawrence, Ottawa (CA)

(72) Inventor: Joseph Lawrence, Ottawa (CA)

(21) Appl. No.: 16/313,318

(22) PCT Filed: Jun. 27, 2017
(86) PCT No.: PCT/CA2017/050783

§ 371 Date: Dec. 26, 2018

(30) Foreign Application Priority Data
Mar. 27, 2017 (CA) 2962221

(51) Int. Cl. A61F 5/02

(52) U.S. Cl. CPC A61F 5/028; A61F 5/32

(57) ABSTRACT

A tension band is provided comprising multiple straps coupled end to end using tension control systems. The tension control systems comprise a tension adjustment mechanism, such as a reel, and a tension member, such as a cable which spans the coupled ends of straps. Each tension control system can be operated independently of the other to draw together or separate further apart, the coupled ends of straps, in order to increase or decrease band tension and thereby selectively apply more or less pressure to different regions of the body part the tension band is wrapped around. The tension bands disclosed may be used around the head, pelvic/hip (sacroiliac) region or lower (lumbosacral) back region of a subject in need of pain relief and to provide support and/or strengthen those areas of the body.

### FIELD OF THE INVENTION

The present invention relates to the field of tension bands to relieve physical pain and discomfort, such as lower back and head pain.

### BACKGROUND OF THE INVENTION

This background to the invention provides information about the state of the art relating to the relief of body pain, and more particularly head and lower torso body pain, such as in the sacroiliac and lumbosacral regions of the back.

While there are various tension bands available on the market, for the most part these products are constructed to rely on the use of the primary closure means for securing the tension band to the body, and to adjust the fit of the tension band to provide more or less support and some pain relief. These bands, however, generally do not allow for selective pressure applications to regions of the body part they are wrapped around.

U.S. Pat. No. 6,554,787 provides a headband to relieve headaches comprising a belt on a first end and a loop on a second end, wherein the headband can be cinched around the head to provide a desired degree of pressure by passing the belt through the loop. Additional headache relief is provided by the inclusion of one or more inner pockets into which heating, cooling, magnetic and vibratory elements can be inserted. Apart from the means of securing the headband to the head of a subject, there is no way to adjust the tension of the headband around the head of the subject without having to undo and re-secure the headband.

An example of a means independent from the primary closure for changing the girth of a band, is disclosed in EP Patent No. 0796568 B1 which provides an alternative adjusting mechanism for headbands with particular application to the inside of helmets and masks. The single adjusting mechanism is configured to facilitate securing and maintaining the band in place around the head and not for the application of pressure to the head to relieve pain or discomfort. The ends of the band overlap and have open slits with teeth that can engage with an adjusting knob comprising a cogwheel operatively connected to a gripping part, so that the band can be adjusted to fit securely around the circumference of the heads of different users. A disadvantage of this band adjusting mechanism is the ability for the hair of a subject to get caught in the band.

US Publication No. 2015/0151070 provides a closure mechanism for securing a headband or mask to the head of a user, which closure mechanism includes a cable and reel system. This system allows the user to perform a one-time macro adjustment or initial fitting, and then micro adjust the fitting of the headband or mask (without undoing the macro fitting), using a single closure mechanism to keep the headband or mask comfortably and securely positioned on the user's head. This publication, however, does not provide for the selective or finer control of pressure applied to different regions of the head for the relief of head pain.

U.S. Pat. No. 9,259,056 provides a single lacing system designed to selectively adjust the size of an opening of an object, such as a belt, which includes a reel, a cable and cable guide configured to allow a user to control the fit of the belt around their body. This patent only discloses the use of a single lacing system to help secure the object and does not disclose applications of the lacing system for the relief of pain by applying pressure around the head, or back of a subject.

US Publication No. 2015/0126918 and US Publication No. 20150059206 provide spine braces with a cable tensioning system including a pair of conformable waist belts which are attached posteriorly near the spine by a single lacing system. The lacing can be tightened or loosened using one or two cable reels such as those sourced from Boa Technology Inc. and disclosed in U.S. Pat. Nos. 7,954,204 and 7,992,261. While this type of spine brace provides for the micro adjustment and control of compressive forces around the waist (thoracolumbar area) of the user, it does not allow for the selective control of anterior and posterior pressure to the pelvis (sacroiliac level) or lower back (lumbosacral junction) of a subject. The design of the brace provided also poses challenges for the cinching of clothing and may accordingly impact the effectiveness of tension adjusting efforts by the user.

US Publication No. 2014/0257156 and U.S. Pat. No. 9,248,040 disclose spine braces with a similar configuration as the brace of US Publication No. 2015/0126918, except that the cable reel is motorized to facilitate the micro adjustment of the brace(s) around the waist of a subject.

Accordingly, there remains a need for a tension band to relieve pain and other physical discomfort, such as headaches and pain in the lower back and pelvic regions of the body. To provide simple and effective pain relief an individual must be able to independently, and selectively adjust the degree of pressure applied to the body part.

### SUMMARY OF THE INVENTION

It is an object of the present invention to provide tension bands for the application of selective pressure to different regions of the body of a subject in order to relieve pain and discomfort in said regions (e.g. head, lower back). The selective control of pressure is achieved by constructing a segmented band wherein two or more segments have an independently operable tension control system which can be readily operated by the subject wearing the band.

Accordingly, the tension band generally comprises multiple straps coupled end to end using tension control systems. The tension control systems comprise a tension adjustment mechanism, such as a reel, and a tension member, such as a cable which spans the coupled ends of straps. Each tension control system can be operated independently of the other to draw together or separate further apart, the coupled ends of straps, in order to increase or decrease band tension and thereby selectively apply more or less pressure to different regions of the body part the tension band is wrapped around. The tension bands disclosed may be used around the head, pelvic/hip (sacroiliac) region or lower (lumbosacral) back region of a subject in need of pain relief and to provide support and/or strengthen those areas of the body.

In one aspect, there is provided a tension band comprising:
three or more straps configured to form a single band, wherein an end of a first strap is coupled to an end of a second strap by a first tension control system, and the other end of the second strap is coupled to an end of a third strap by a second tension control system; and
a closure system for securing the free ends of the band around a body part of a subject,
wherein when the band is secured around the body part of a subject, each tension control system can be independently operated by the subject to adjust the degree of pressure applied by the band to the body part. Exemplary uses of the tension band for a body part, may include the head, pelvis or lower back of a subject.

In one embodiment of the tension band, each tension control system comprises a tension adjustment mechanism operatively associated with a tension member which spans the coupled ends of two straps, wherein the subject uses the tension adjustment mechanism to change the degree of pressure applied by the band to the body part by adjusting the length of the tension member that spans the coupled ends of two straps.

In another embodiment of the tension band, the tension member is a cable that spans and is affixed proximal to the coupled ends of straps, and the tension adjustment mechanism is affixed proximal to one of the coupled ends of said two straps. In a related embodiment, the ends of the cable are housed within the tension adjustment mechanism.

Exemplary uses of the tension band for a body part, may include the head, pelvis or lower back of a subject.

In another aspect there is provided a tension band comprising:
three or more straps configured to form a single band, wherein an end of a first strap is coupled to an end of a second strap by a first tension control system, and the other end of the second strap is coupled to an end of a third strap by a second tension control system; and
a closure system for securing the free ends of the band around the head, pelvis, or lower back of a subject,
wherein each tension control system comprises a cable that spans and is affixed proximal to the coupled ends of two straps, said cable being operatively associated with a tension adjustment mechanism affixed proximal to one of the coupled ends of said two straps; and
wherein when the band is secured around the head, pelvis, or lower back of a subject, each tension control system is positioned substantially to the side of the head, pelvis, or lower back and can be independently operated by the subject to adjust the degree of pressure applied by the band to the head, pelvis, or lower back by using the tension adjustment mechanism to adjust the length of the cable spanning the coupled ends of two straps.

In still a further aspect a use of a tension band for a body part is provided, the tension band comprising:
three or more straps configured to form a single band, wherein an end of a first strap is coupled to an end of a second strap by a first tension control system, and the other end of the second strap is coupled to an end of a third strap by a second tension control system; and
a closure system for securing the free ends of the band around a body part of a subject,
wherein when the band is secured around the body part of a subject, each tension control system can be independently operated by the subject to adjust the degree of pressure applied by the band to the body part. Exemplary uses of the tension band for a body part, may include the head, pelvis or lower back of a subject.

### BRIEF DESCRIPTION OF THE DRAWINGS

These and other features of the invention will become more apparent in the following detailed description in which reference is made to the appended drawings.

FIG. 1: is a top view of an embodiment of a tension band for the head (headband) of a subject. The exemplary lengths of different regions of the tension band are shown millimeters.

FIG. 2: is a top view of an embodiment of a tension band for the pelvis/hips (belt) of a subject. The exemplary lengths of different regions of the tension band are shown millimeters.

FIG. 3: is a top view of an alternative embodiment of a headband designed to wrap around the sub-occipital region of a subject's head. The exemplary lengths of different regions of the tension band are shown millimeters.

FIG. 4: is a view of the headband of FIG. 1 around the head of a subject.

FIG. 5: is a view of the belt in FIG. 2 around the hips of a subject.

FIG. 6: is a side view of the embodiment of the headband shown in FIG. 3 on the head of a subject.

FIG. 7: is an isometric view of a tension band padding accessory with a single pressure pad affixed to a tension band for the pelvis/hips of a subject.

FIG. 8A: illustrates a bottom view of an embodiment of a tension band for the pelvis/hips of a subject with a topside view of a tension band pad accessory affixed to it.

FIG. 8B: illustrates a top view of the tension band for the pelvis/hips (belt) of a subject with an underside view of a tension band padding accessory affixed to it.

FIG. 9: is a view of the underside of a tension band padding accessory.

FIG. 10A: is an isometric view of a tension band padding accessory with two pressure pads affixed to a tension band for the pelvis/hips of a subject.

FIG. 10B: is a bottom view of an embodiment of a tension band for the pelvis/hips of a subject with a topside view of the tension band padding accessory shown in FIG. 10A.

FIGS. 11A and 11B: illustrate side and underside views, respectively, of the single pressure pad shown in FIGS. 7 and 8A.

FIGS. 11C and 11D: illustrate side and underside views, respectively, of the two pressure pads shown in FIGS. 10A and 10B.

### DETAILED DESCRIPTION OF THE INVENTION

The present invention relates to the field of tension bands to relieve physical pain and discomfort, such as lower back and head pain. A subject is provided with a simple and effective means for selectively controlling the degree of pressure applied or felt around the head, pelvis or lower back regions when using the tension band. The tension band is comprised of multiple strips of material (straps) coupled end to end to form the tension band. The coupling of straps is facilitated using a tension control system that spans the ends of the straps coupled to one another and which can be used to bring the ends together, separate them, or change the degree of overlap between them. The use of multiple tension control systems to couple straps of a tension band allows a subject to change the tension around the body part differentially and selectively relieve pain felt at different regions of the body part.

### Definitions

Unless defined otherwise, all technical and scientific terms used herein have the same meaning as commonly understood by one of ordinary skill in the art to which this invention belongs.

The terms “relief”, “relieve” or “relieved” as used herein, refer to an intervention performed with the intention of alleviating the symptoms associated with, or preventing the development of pain or discomfort in a region of the body of a subject, as well as altering the pathology of a disease, disorder or condition. Thus, the terms used in their broadest sense, and in various embodiments include one or more of the prevention (prophylaxis), moderation, reduction, and/or curing of the condition giving rise to the physical pain or discomfort felt by a subject. Subjects in need of pain relief, are those already having a disease, disorder or condition as well as being prone to, or at risk of developing, the disease, disorder or condition and those in whom the disease, disorder or condition is to be prevented.

The terms “subject” and “patient” as used herein both refer to a human or non-human animal. In certain embodiments, the subject or patient is a human.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to.

As used herein, the terms, “couple”, “coupled” and “coupling” refer to the functional aligning of an end of one strip of material to an end of another strip of material in proximity to one another, and in a manner in which the proximity of said ends may be adjusted using a coupling means to separate said ends, or bring them closer together. The coupled ends of strips of material may be permanently or semi-permanently coupled, as well as capable of being selectively uncoupled depending on the choice of coupling means applied. The functional aligning of said ends of strips of material may include configurations where there is a gap between coupled ends of strips of material, or configurations where strips of material overlap one another.

As used herein, the term “affixed” refers to the direct or indirect attachment of a component or mechanism to a strap or strip of material, such as a tension adjusting mechanism or tension member.

As used herein, the term(s) “strip(s) of material” or “strap(s)” interchangeably refer to a continuous length of a material (e.g. a textile strap) incorporated into a tension band. Each strip of material or strap comprises one or more pieces of textile and/or components that are permanently or semi-permanently attached in order to form and function as a continuous length of material. When one or more pieces of textile material and/or components are used to form a strap, they may be attached or bonded by various means such as, but not limited to sewing, fusing, and gluing, or otherwise attached using rivets, snaps, hooks, zippers and the like. When the tension band is in use to help relieve the pain and discomfort of a subject, the various pieces of textile and/or components forming a single strip of material are intended to remain attached, but when the tension band is not in use, one or more pieces of textile material and/or components may be detached to replace or repair the strip of material to restore optimal functionality to the tension band, e.g. to sew a torn textile, replace a faulty zipper, or secure a hook or snap that has become loose.

As used herein the terms “segment” or “region” refer to part of a tension band or body part. In the case of a reference to a segment or region of a tension band, the segment or region need not correlate to a strap of a tension band and may include short or long segments/regions which span part of a strap or which span two or more straps, as the case may be.

As used herein the term “pressure” refers to a compressive force applied to, against and/or felt by a subject on a part of their body.

As used herein the term “tension” refers to a tensile force applied to or in, one or more regions or segments of a tension band wrapped around a body part of a subject and resulting in the subject feeling more pressure (compressive force) on, at or around a part of their body. The release of tension at or in one or more regions or segments of the tension band relieves the pressure felt by a subject on, at or around a part of their body. A “tension control system” is to be understood in a broad sense, to refer to one or more elements or features operatively associated to control the degree of tension applied to a region or segment of a tension band. A “tension adjustment mechanism” is to be understood in a broad sense, to refer to one or more elements or features operatively associated to implement a change in tension when operated by a user. A “tension member” is to be understood in a broad sense, to refer to the element(s) or feature(s) that the tension adjustment mechanism acts upon to implement a change in tension to or in a region or segment of the tension band.

As used herein the term “head” may be understood to refer to the entire head or parts thereof, including the forehead, occipital and sub-occipital regions of the head.

As used herein the term “pelvis” or “hips” refers to the region of the body around or at the sacroiliac joint level of the spine.

As used herein the term “lower back” refers to region of the body around or at the lumbosacral junction of the spine.

It is contemplated that any embodiment discussed herein can be implemented with respect to any disclosed method, use, device or composition, and vice versa. Furthermore, devices and compositions of the invention can be used to achieve the disclosed methods and uses.

While the following description and the figures detail certain embodiments to illustrate and exemplify the invention, it is to be understood that the invention is not limited by the details of the construction and specific illustration of such embodiments which follows.

### Tension Band Construction

Embodiments of the invention relate to tension bands constructed for application to different body parts and which may provide one or more functionalities to different regions of a body part, in addition to the selective control of pressure applied to, or felt by a user (subject) on or at different regions of said body part.

In one embodiment a tension band is constructed for use around the head of subject. In a related embodiment a tension band is constructed to apply pressure to the occipital region of a head. In another related embodiment a tension band is constructed to apply pressure to the sub-occipital region of a head.

In another embodiment a tension band is constructed for use around the pelvis or hips of a subject, to provide relief from pain and discomfort.

In a further embodiment a tension band is constructed for use around the lower back of a subject, to provide relief from pain and discomfort.

In yet another embodiment, a tension band is constructed for use to provide support to the pelvic and/or lower back regions of a subject.

In still a further embodiment, a tension band is constructed for use to strengthen the pelvic and/or lower back regions of a subject.

### Tension Band Material and Dimensions

The tension bands provided herein are constructed of three or more strips of material (straps) arranged end to end to form a single band. Each strip of material may be constructed of one more pieces of flexible and/or semi-rigid textile, such as fabric, cloth, ribbon and other woven fibres, netting, mesh, natural and synthetic leathers, synthetic rubbers or rubber-like materials (e.g. Neoprene), foam (e.g. foam swatches), and/or other components, such as, plastics, paddings, linings, waddings, Velcro™ patches/strips, pins, hooks, snaps, ties, elastics, ligatures, zippers, and other fastening means, and combinations of the foregoing.

Textiles and/or components may be sewn, glued, bonded, fused, riveted, snapped, clipped, hooked, zipped, or otherwise attached to form and function permanently or semi-permanently as a single strip of material. The tension band may be constructed to facilitate the repair or replacement of materials and/or components to restore optimal functionality. Suitable textiles and components (e.g. medical grade) may be selected by one skilled in the art to provide different regions of the band with different properties or functionalities and properties, such as comfortable fit, support, resistance to wear and tear, mould, mildew and bacteria, and hypo-allergenic properties, in addition to the ability to adjust the degree of pressure applied to or felt by a subject on a body part.

When the straps are attached end to end, the construction of the band should be sufficiently flexible for the band to adopt a generally annular form around the head or hips of a subject and conform to the shape of the body part. The side of the band next to and facing the body part it is wrapped around, should have substantially uniform contact with the body part of the subject when fitted securely around said body part. It will be understood by one skilled in the art that “contact with the body part” may be direct or indirect depending on whether the user subject is wearing clothing or apparel at the body part around which the tension band is wrapped.

In one embodiment, the tension band may be incorporated into the clothing or apparel worn by a subject. In a related embodiment, the tension band may be incorporated into a body suit or sport apparel. In another related embodiment, the tension band may be incorporated into a ball cap or other head apparel, such as a sun hat.

One skilled in the art would be able to select suitable combinations or textiles and/or components to construct the straps for tension bands provided for herein. Exemplary combinations of textiles and/or components for the construction of straps incorporated into the tension bands of the present disclosure, are disclosed in U.S. Pat. No. 6,554,787.

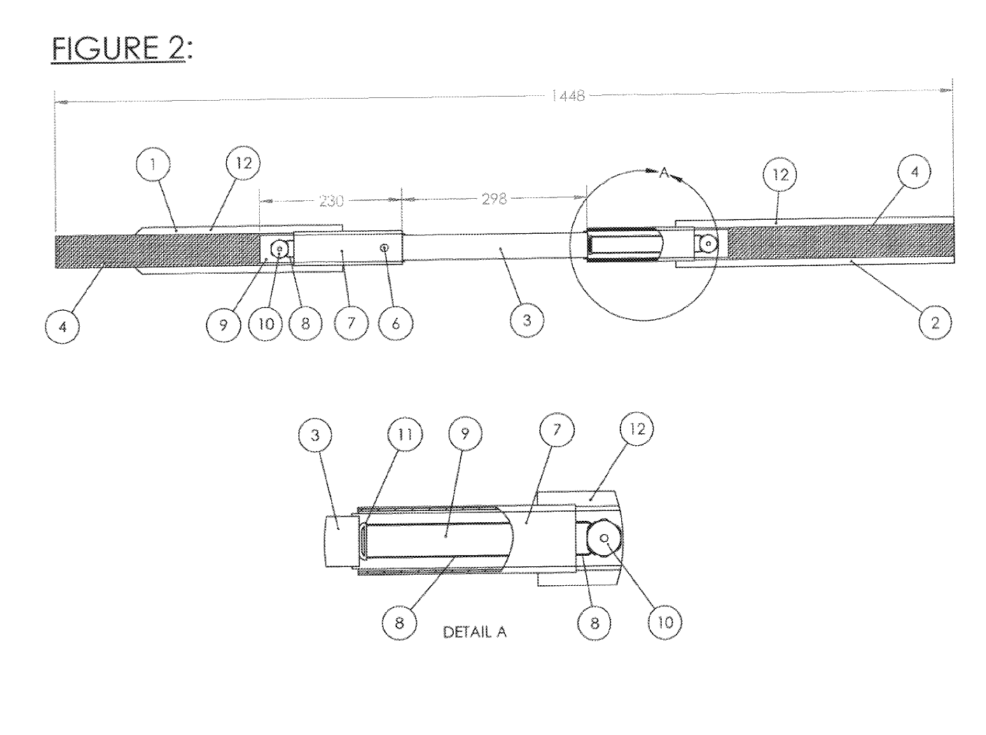

In one embodiment, a strip of material or strap of a tension band comprises one or more pieces of textile. In a related embodiment, the pieces of textile are layered in whole or in part relative to one another. As illustrated in FIG. 2, straps 1 and 2 comprise Velcro™ strips 4 and a textile layer 12.

In another embodiment, a strip of material or strap of a tension band comprises a textile with elastomeric properties.

In still another embodiment, a strip of material or strap of a tension band comprises one or more textiles and/or components which function as padding.

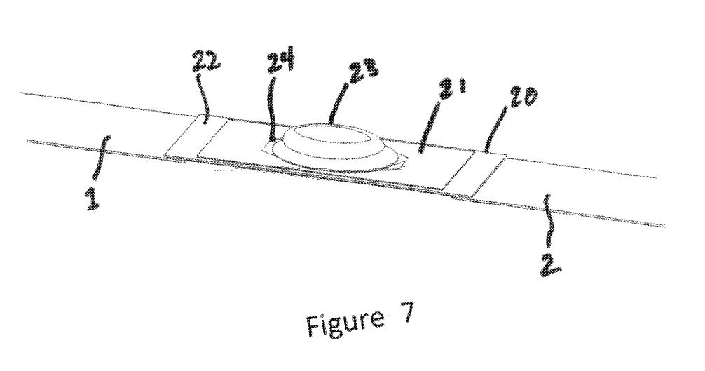

In a related embodiment, a strip of material with padding may be permanently or removably affixed as an accessory, or addition to the tension band. The padding accessory can be affixed to a strip of material/strap of the tension band using a variety of attachment means (e.g. Velcro™, belt loops, stitching, etc.), on the side of the tension band that will be up against the body of the user when the band is in use. Pressure padding can be permanently or removably affixed to a flexible (e.g. stretchable or elastic) and/or reinforced padding accessory strap as shown in FIGS. 7-11. The padding (back pads) of the accessory may be made of standard, medical grade, foam materials, such as, but not limited to polyethylene or polyurethane. Materials such as these are known to be resilient, non-abrasive, and highly resistant to solvents, mildew and bacteria.

The padding may be shaped and placed to apply pressure at specific points at the back of the body at or near the lumbosacral and sacroiliac regions of the user's back while at the same time not substantially affecting the functionality of the tension band around the rest of the body. The pressure pads help to alleviate pain, while the supporting strip of material (padding accessory strap) is of a semi rigid construction or sufficiently stiff to support the functionality of the pressure pad(s) and also serve as a further guide surface over which the strips of the tension band can move (e.g. glide) when the user is adjusting the tension band using one or more of the tension control systems. The use of a padding accessory with the configurations shown in the figures and described in Example 3 thereby assist to prevent the user's clothing from bunching up when the band is being adjusted.

In yet another embodiment, a strip of material or strap of a tension band comprises a Velcro™ fastening means.

In a further embodiment a strip of material or strap of a tension band may comprise one or more pockets or compartments.

In still a further embodiment a strip of material or strap of a tension band comprises one or more textiles with moisture wicking properties.

In yet a further embodiment, a strip of material or strap of a tension band comprises one or more textiles and/or components with hypo-allergenic properties.

In one other embodiment, a strip of material or strap of a tension band comprises one or more textiles and/or components with insulating properties.

The tension bands may be constructed in different dimensions to accommodate a variety of body parts and body types. The length of the tension band may be custom made for an individual or sufficiently long and adjustable to accommodate a variety of individual sizes.

In one embodiment, a tension band is sufficiently long to wrap around a body part of a subject and for its length to be adjustable for a total of between about 4 to about 14 inches. In a related embodiment, the tension band is adjustable for up to a total of between about 6 to about 13 inches. In still another related embodiment, the tension band is adjustable for up to a total of between about 8 to about 12 inches. In yet another embodiment the tension band is adjustable for up to a total of about 4 to about 8 inches. In still another embodiment the length of the tension band can be adjusted by up to about 12.8 inches.

In one embodiment, each tension control system can adjust the length of the tension band in equal proportions. In a related embodiment, each tension control system can adjust the length of the tension band by about 2, 4 or 6 inches. In another related embodiment, each tension control system can adjust the length of the tension band by about 6.4 inches. In an alternative embodiment, different tension control systems can adjust the length of the tension band in unequal or different proportions. These embodiments allow for the customized construction of tension bands and for the differential application of pressure to different regions of the body part it is wrapped around depending on the needs of a subject.

In another embodiment, the length of the tension band may be made to adjust to fit the hips of a subject ranging in size from about 35 to about 50 inches around.

Alternatively, in another embodiment a tension band for the hips may be constructed in multiple lengths or sizes, such as about 49 inches (small/medium), about 53 inches (medium/large) and about 57 inches (large/extra large) in its fully extended state. In a related embodiment the length or size of the tension band may be adapted with the use of separate strap wherein each end can be secured to the free ends of the tension band. The use of such an additional strap to lengthen and secure the tension band provides the subject with more band girth as needed and allows the subject to continue to use the same tension band while accommodating changes in hip size.

In another embodiment, the length of a tension band for a head is about 725 to about 750 millimeters when the tension band is in it most extended state. In a related embodiment, the length of the tension band for the head is about 733 millimeters, when the tension band is in it most extended state. In another embodiment the tension band for the head is made to fit and be sufficiently adjustable to alleviate headache pain for heads with a circumference ranging from about 56 cm to about 64 cm.

The width of the tension band at any given point should be sufficient to provide a base for the attachment of a tension control system at or near the junction points where the ends of strips of materials are coupled. The width of the tension band may also be adapted to be different widths for different band segments or regions to ensure a comfortable and secure fit, as well as provide other functionalities for different points of the body, such as support.

In one embodiment the tension band is at least one inch (25 millimetres) wide at any given point along its length. In another embodiment the width of the tension band is at least about 2 inches wide at any given point along its length. In yet another embodiment the width of the tension band is wider along the stretches or segments of the band where the application of pressure will be more acute when the band is in use. In still a further embodiment the width of the tension band may vary to accommodate the placement or incorporation of additional components, the tension control system and/or closure system. As shown in the illustrative embodiment of a tension band in FIG. 2, the widths of the straps of the tension band range from about 2 to about 3 inches along different segments according to where the tension control systems and Velcro™ closure means are placed.

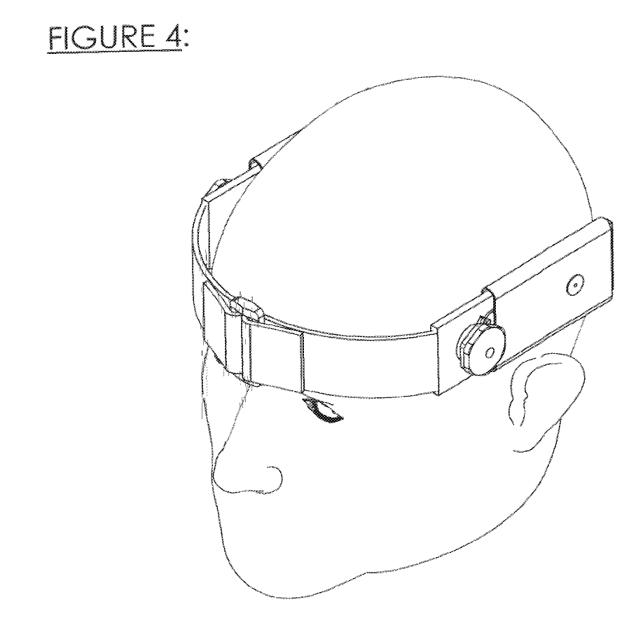

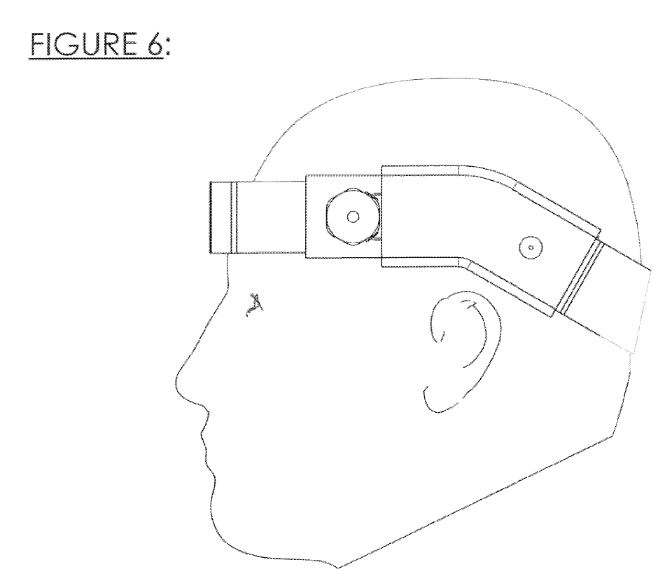

The configuration of the strips of material coupled end to end to form the tension band may be configured at a substantially 180° (linear) angle relative to one another, or the strips of materials may be coupled at a non-linear angle relative to one another to allow the tension band to fit to or around different regions of a body part. In one embodiment, the tension band is a headband where the strip of material at the back of the head fits around the occipital region of the head, as shown in FIG. 4. In another embodiment, the tension band is a headband where the strip of material at the back of the head fits around the sub-occipital region of the head, as shown in FIG. 6.

### Tension Control System

A tension control system is used to couple the ends of strips of material to form the tension bands provided herein. The tension control system comprises a tension member and tension adjustment mechanism used to adjust the distance between coupled ends of strips of material and thereby change the pressure applied to a body part or felt by a subject wearing or fitted with a tension band. The application of tension control systems at the juncture points linking the ends of strips of material, allows for the fit and pressure exerted by the band around a body part of a subject to be adjusted without having to undo the band once macro fitted and secured around said body part.

Various suitable tension control systems for coupling the ends of strips of material are known and may be selected by one skilled in the art, such as the tension control systems disclosed in U.S. Pat. Nos. 6,554,787 and 9,259,056, and US Publication Nos. 2015/0151070, 2015/0126918 and 20150059206. The tension control systems suitable for coupling the ends of two straps in the tension bands disclosed herein, all allow for the micro adjustment of tension in the tension band and accordingly the micro adjustment of pressure felt by the subject using the tension band. The use of multiple (two or more) tension control systems in a tension band allows for more selective and finer control over the application of pressure at different regions of the body part the band is wrapped around.

In one embodiment the tension band is configured with tension control systems to apply anterior and posterior pressure to the body part the tension band is wrapped around. In another embodiment, use of a tension control system incorporated into a tension band and positioned on the left side of a body part, relieves pain felt by a subject on the right side of the body part. In a further embodiment use of a tension control system incorporated into a tension band and positioned on the right side of a body part, relieves pain felt by a subject on the left side of the body part.

In one embodiment, the tension control system comprises a tension member and a tension adjustment mechanism. In a related embodiment, the tension member is a cable and the tension adjustment mechanism is a reel. The cable and reel are operatively associated such that the cable can be wound and unwound around a spool of the reel. The reel includes a tension adjusting means such as a gripping member (e.g. dial or knob) or handle member for turning the reel.

In a related embodiment, the tension control system comprises a low power IP1 reel system. The IP1 reel system can be sourced from Boa Technology Inc. Each IP1 reel system includes a dial (knob), spool and lace (cable).

In yet another embodiment, the reel of a tension control system may be turned clockwise and counter-clockwise to wind or unwind the cable. In still another embodiment, the reel may be selectively enabled or disabled to facilitate the winding and unwinding of the cable and set the desired degree of tension in the band. In a related embodiment, the reel may be selectively locked or unlocked to adjust and sustainably maintain the desired degree of tension in the band. Locking and unlocking the reel may be achieved by lifting the tension adjusting means (e.g. a knob or dial) to disengage it from the rest of the tension adjusting mechanism and snapping it down so that it engages the rest of the tension adjusting mechanism when in use.

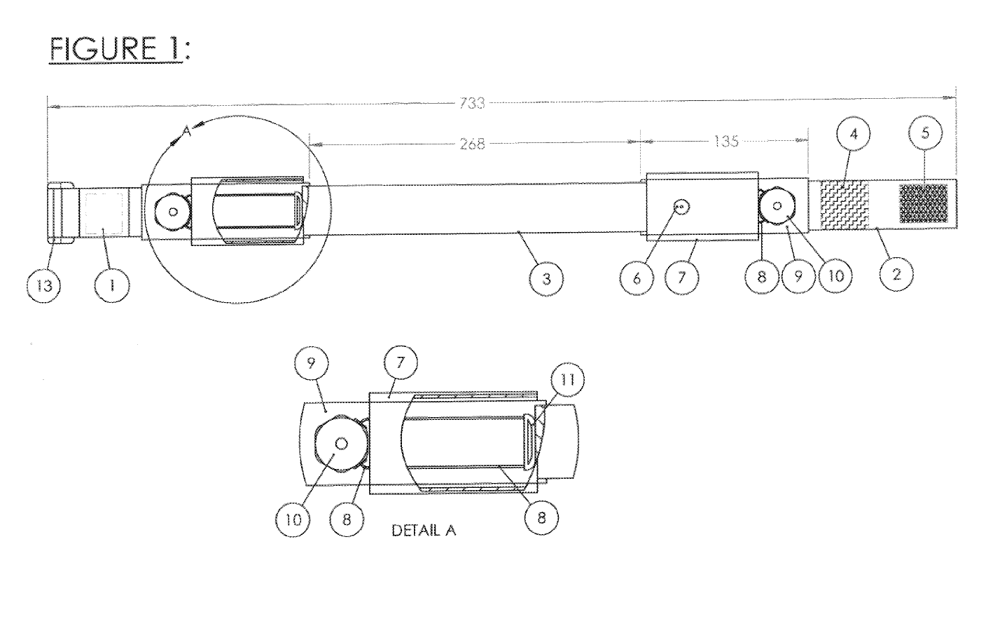

In one embodiment, each tension control system incorporated into a tension band is configured to change the overall length of a tension band by about 1 to 2 centimeters with each full turn of a dial or knob (tension adjusting means) forming part of a reel 10, as shown in FIGS. 1-3. In a related embodiment one full turn or cycle of the tension adjusting means in a given direction changes the length of the tension band by about 1.5 centimeters (to make it longer or shorter). In another embodiment, each tension adjusting means forming part of a given tension adjustment mechanism is configured to change the overall length of a tension band to different degrees.

The tension adjustment mechanism can be designed to meet the needs of subjects who may have limited manual dexterity and strength (e.g. due to arthritis or Parkinson's disease). For example, in the case where the tension adjustment mechanism comprises a reel, the configuration of said reel may be set so that a user can turn the spool of the reel using a dial or knob a short distance with little resistance to effect the micro-adjustment of the cable.

In a further embodiment, the cable of the tension control system spans and is movably affixed directly or indirectly to the coupled ends of two strips of material. Various materials and or components can be used to protect the cable from being cut or damaged. In one embodiment the tension control system is housed in a sleeve member. The sleeve member is sized to easily allow for the coupled ends of two straps to slip into or through it. For example, as illustrated in FIG. 2 the sleeve member 7 is about 2¼ inches wide to receive strap ends that are 2 inches wide.

In another embodiment, the tension adjustment mechanism is mounted on a semi-rigid backing which spans the coupled ends of two strips of material. The backing may be rectangular and long enough to provide additional protection for the tension control system and serve as a guide for the movement of the ends of two coupled straps as they are brought together or separated using the tension adjustment mechanism. Illustrative backings 9 are shown in FIGS. 1 and 2. Alternatively, the backing may be square to simply serve as a mount for the reel or other tension adjustment mechanism. In one embodiment of a tension band for the hips, the reel is mounted on a backing that is about 3 inches square.

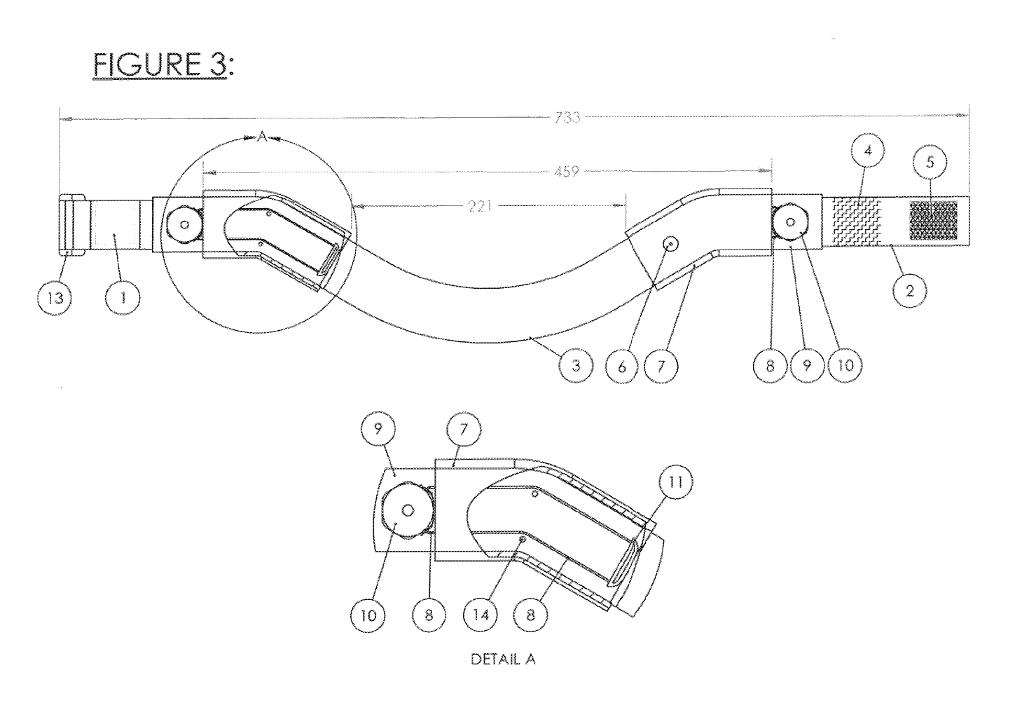

In still a further embodiment, the cable is bi-directionally guided around the spool of the reel near or proximal to one end of a first strap and along a first guide member attached near or proximal to the end of a second strap coupled to the end of the first strap. In yet a further embodiment, the cable may pass through, along or around one more additional guide members in between the reel and the first guide member. In a related embodiment, one or more additional guide members are positioned to facilitate the coupling of straps at a non-linear angle relative to one another (e.g. see FIG. 3). One skilled in the art will readily be able to select and implement appropriate placements and combinations of guide members to implement the required lacing pathway for a cable, as exemplified in US Publication No. 2015/0059206.

In one embodiment the tension control system allows for the adjustment of tension by winding and unwinding the cable in increments less than one millimeter. In another embodiment the tension control system allows for the adjustment of tension by winding and unwinding the cable in increments of one millimetre. In yet another embodiment, the tension control system allows for the adjustment of tension by winding and unwinding the cable in increments greater than one millimetre.

In a further embodiment a tension control system may be operated by the subject using one hand, when the subject is wearing the tension band.

In an alternative embodiment, a tension control system which couples the ends of two strips of material in a tension band, may be configured such that the tension adjustment mechanism can be removably or semi-permanently affixed (e.g. snapped onto) the end of strip of material to facilitate the coupling and uncoupling of strips of material. The ability to couple and decouple strips of material used in the tension bands provides an alternative means for the subject to put on and take off the band without changing the macro adjusted girth of the band when initially fitted around a body part of the subject. Exemplary tension control systems which provide this functionality are disclosed in US Publication Nos. 2014/0123440 and 2015/0151070.

### Band Closure System

The tension bands provided herein comprise a closure system for securing the band around a body part of a subject. Exemplary means for securing a band around a body part of a subject are disclosed in U.S. Pat. Nos. 6,554,787 and 9,259,056, and US Publication Nos. 2015/0151070, 2015/0126918 and 20150059206.

In one embodiment, the closure system of the tension band comprises one or more Velcro™ strips or patches proximal to the free ends of the band. Complimentary patches are positioned respectively on opposite ends of the tension band in order to fasten, unfasten and macro fit the tension band around a user's body. In an embodiment using an extra strap to link the free ends of the band, Velcro™ strips or patches may similarly be used to connect the free ends of the band to the additional strap used to lengthen the band and provide more girth when in use by a subject.

In another embodiment, the closure system of the tension band is a belt-like buckle system.

In a further embodiment, the closure system of the tension band comprises a ring structure. In a related embodiment the ring structure is permanently or semi-permanently attached to one end of the tension band. To macro fit the tension band around the user's body, the other end of the tension band is passed through the ring structure and looped around the ring back on itself to secure the band using a suitable fastening means. In another related embodiment, either or both ends of the tension band (at the user's election) can be run through, looped around the ring structure to fasten (and unfasten) the tension band and to macro fit the band securely around the user's body.

In yet another embodiment, the closure system of the tension band comprises a tension control system in which the tension adjustment mechanism may be removably attached and detached from one end of the tension band.

In a further embodiment, the closure system is used to macro fit the tension band around the body of a subject when the tension band is at its maximum extended length. In still another embodiment, the closure system is used to macro fit the tension band around the body of a subject when the length of the tension band is first partially adjusted using one or more tension control systems.

### Tension Band Uses

Exemplary uses for the tension bands provided herein are for the relief of pain and discomfort in the head, pelvic and lower back regions of the body.

In one embodiment, the tension band is used for the treatment of a patient under the supervision of a medical professional.

In another embodiment, the tension band is used independently by a subject.

In still another embodiment, the tension band is a headband used to alleviate the pain associated with a tension-type headache in a subject.

In yet another embodiment, the tension band is a belt used to alleviate the pain associated with the lumbosacral and sacroiliac regions of the body of a subject.

In a further embodiment, the tension band is used to provide support to the pelvic and/or lower back regions of a subject.

In still a further embodiment, the tension band is used to strengthen the pelvic and/or lower back regions of a subject.

In a related embodiment, the tension band is incorporated into head or body apparel worn by a subject.

The following examples of tension bands and uses thereof are set forth and described with reference to the Figures. It will be understood that these examples are intended to describe illustrative embodiments of the tension bands and uses thereof provided herein and are not intended to limit the scope of the invention in any way.

### EXAMPLES

### Example 1

### Prototype and Testing of Tension Band for the Head

An exemplary tension band for the head (headband) was constructed with the features shown in FIG. 1. Three strips of material 1, 2 and 3 are provided and coupled to each other by two tension control systems, one of which is shown in detail in DETAIL A of FIG. 1. Strips of material 1 and 2 each have Velcro™ patches 4 and 5 as shown on the strip of material 2, to facilitate closure of the band around the head of a subject by passing each of the ends of the headband through the ring structure 13 and back on itself as shown for the strip of material 1.

Each tension control system comprises a reel 10 attached to a backing 9 which is attached to or near the coupled end of the strips of material 1 and 2. Each reel 10 is operatively associated with a cable 8. The two ends of the cable 8 are affixed in the reel 10 where there is a spool (not shown) to facilitate the winding and unwinding of the cable when the reel is operated by a user. The cable runs around or along a guide member 11 affixed to a coupled end of the strip of material 3.

In an alternative embodiment, the reels 10 may be attached (directly or indirectly via a backing) to the ends of the strip of material 3 and the cable guides 11 attached to the ends of the strips of material 1 and 2.

Each tension control system is housed within a sleeve 7 to protect the cable and the sleeve 7 is attached to the backing 9 using a rivet 6.

Each reel 10 may be independently operated such that when a reel 10 is turned in either direction to wind or unwind the cable 8, the coupled ends of the strips of material are guided along the backing 9 and brought closer together or separated, to increase or release tension in the band, accordingly.

An illustration of how the headband of FIG. 1 may be fitted on a subject is shown in FIG. 4.

The tension band shown in FIG. 1 was configured to wrap around the occipital region at the back of the head and was tested on at least 35 subjects with head pain ranging in location from the forehead to the temporal region to the apex of the skull as well as the sub-occipital region. The headaches of the subjects were significant enough to lead them to self-medicate and/or undergo therapy. The types of headache were most often (about 95%) tension-type headaches, with the balance being migraine headaches. All of the subjects reported no difficulty using the device, and appreciated the ability to quickly control the tension with a simple turn of the knobs of the tension adjusting mechanism. The majority (about 70%) of patients reported significant pain relief ranging from about 20-80%. The remaining about 30% of patients found the band ineffective or uncomfortable. Overall the band was most effective for tension-type headaches and showed some efficacy for migraine type headaches.

An alternative embodiment of a headband is shown in FIGS. 3 and 6. In this embodiment, the headband is configured to wrap around the sub-occipital region of the head. The ends of strip of material 3 are coupled to one end of each of strips of material 1 and 2 at an angle which can range between about of 30-45°. A bent or multi-piece backing may be used to facilitate setting the desired coupling angle for the strips of material, by providing a base for setting additional guide members 14 as shown in FIG. 3, in between the reel 10 and primary (first) guide member 11 used to couple two straps together.

In yet a another variation of the embodiment shown in FIG. 3, the coupled ends of the strips of material 1 and 2 may be configured to bend (at an angle which can range between about 30-45°) to be coupled to the ends of the strip of material 3. In this embodiment, the tension control system may be positioned in alignment with the slope created at the bent ends of the strips of material 1 and 2 if there is less of a draw length or area required for the cable to shorten the tension band. In a further related embodiment, the tension control systems may be positioned to follow the bend of the strips of material 1 and 2, such that two or more guide members 14 are used to guide the cable around the bend in order to span the bent ends of strips of material 1 and 2 coupled to the ends of strip of material 3.

In all of the variations of the tension band specifically and generally illustrated in FIGS. 3 and 6, each strip of material is of a linear construction. Strip of material 3, however, is shown in FIG. 3 in a curvilinear form to illustrate how it is incorporated into the tension band to conform to and cradle the sub-occipital region of the head.

### Example 2

### Prototype and Testing of Tension Band for the Pelvis and Lower Back

An exemplary tension band for the pelvis/hips (belt) was constructed with the features shown in FIG. 2. The configuration of the belt is similar to the headband of FIG. 1, in terms of how the strips of material 1, 2 and 3 are aligned with one another and the positioning and construction of the tension control systems used to couple the strips of material, each comprising a reel 10, a guide member 11 and a cable 8 which spans and couples the ends of two strips of material. The tension control systems are similarly supported by a backing 9 and protected by a sleeve 7. In this embodiment, however, the closure system varies from that of the exemplary headband in FIG. 1, in that it comprises a Velcro™ fastening means 4 (and complementary Velcro™ strips on the other side of the band (not shown), but no ring structure. Accordingly, the user need only bring together complementary Velcro™ strips in order to secure the belt around the hips or lower back.

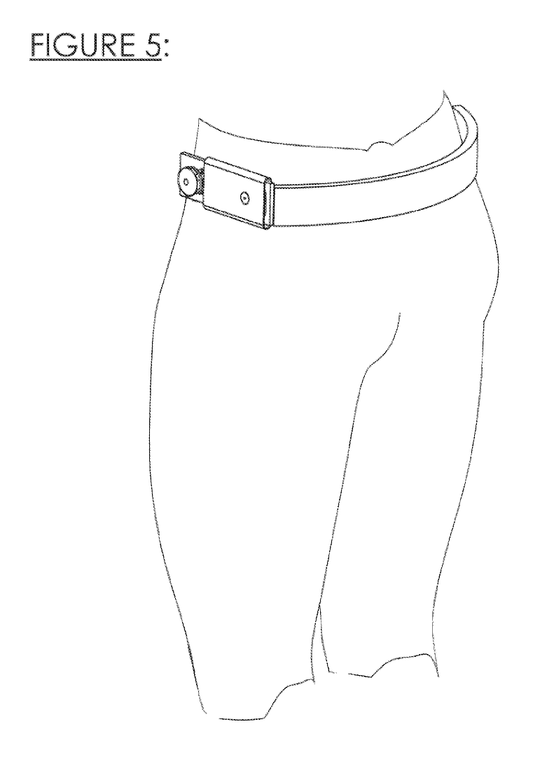

An illustration of how the belt of FIG. 2 may be fitted on a subject is shown in FIG. 5.

The pelvic (sacroiliac) tension band (belt) was tested on over 60 subjects with uncomplicated mechanical low back pain (with or without sciatic radiation) as well as sacroiliac pain. This belt was very well received by users for the ease of use, as well as its ability to provide an adequate amount of support. The ability to micro adjust tension immediately was especially helpful when changing positions, as well as providing more tension on one side versus the other as needed. The quick release was very helpful when the belt needed to be adjusted to a new position on the body, without having to completely remove it. About 90% of patients found relief ranging from about 30-90%. The belt seemed to work very well for pain in the lumbosacral and sacroiliac regions, and showed some but more limited efficacy for very intense sciatic pain. Furthermore, the more patients wore it the less they seemed to need it, as there was apparently benefit for its utility in back strengthening over time.

### Example 3

### Tension Band Padding Accessories

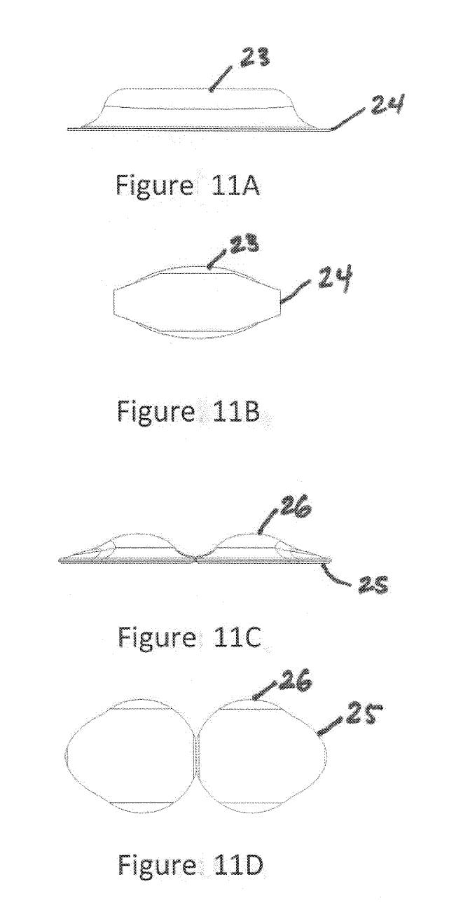

Additional alleviation of pain or discomfort may be achieved by affixing pressure pads on the surface of the tension band that is against the body when in use (bottom side). FIGS. 7 to 11 illustrate various aspects of two embodiments of tension band padding accessories 20 with two different pressure pad configurations. FIG. 7 illustrates a single pressure pad structure 23 affixed to a Velcro™ backing (patch) 24 which may be attached and detached from a complementary Velcro™ strip 21 on the padding accessory strap 22. The pressure pad 23 with its Velcro™ backing (patch) 24 is shown in greater detail in FIGS. 11A and 11B.

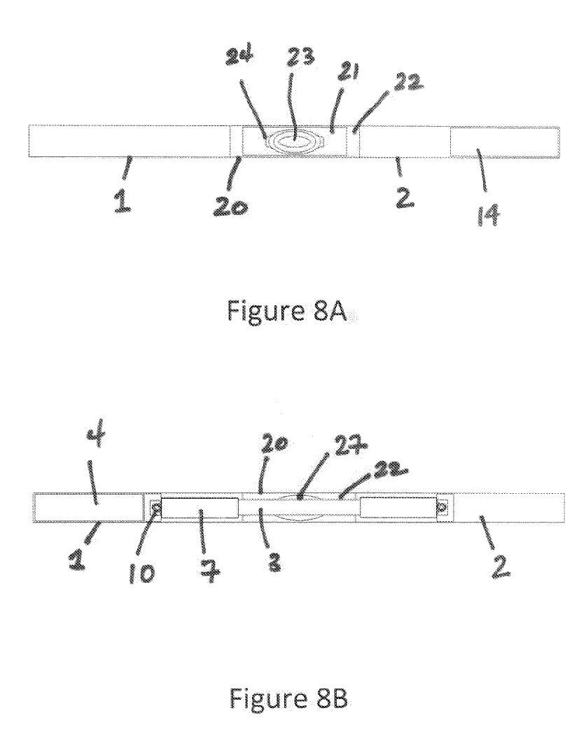

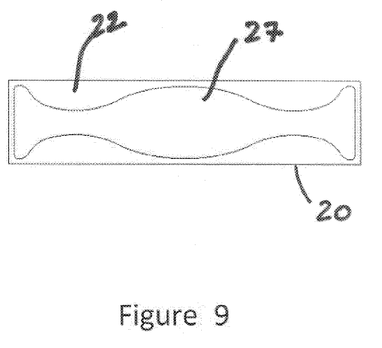

FIG. 8 illustrates the integration of the tension band padding accessory 20 of FIG. 7 with a tension band for the body according to the present disclosure. FIG. 8A illustrates the single pressure pad 23 which will rest against the body when the tension band is in use. FIG. 8B illustrates the underside of the tension band padding accessory 20 which substantially spans the mid strip 3 of the tension band. A plastic stiffening feature 27 is affixed or integrated with the padding accessory strap 22 (shown in greater detail in FIG. 9). The stiffening feature 27 provides various benefits including supporting the pressure pad 23 and its application against the body, as well as the integration of the tension band padding accessory 20 with the tension band. Depending on the shape of the stiffening feature 27, the flexibility and rigidity of the tension band padding accessory 20 can be varied to support the preferred or optimal functionality of the tension band, when a user is wearing the tension band, or adjusting a reel 10 of the tension control system.

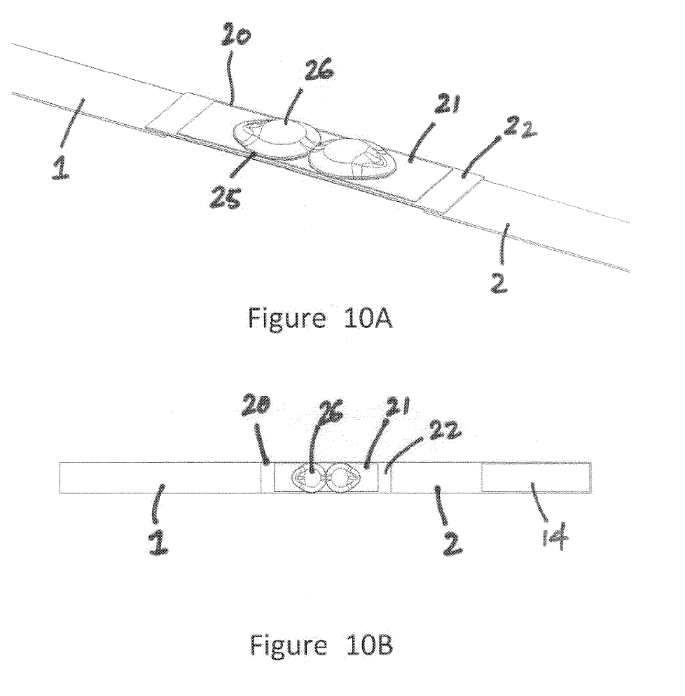

An alternative embodiment of the tension band padding accessory 20 is illustrated in FIGS. 10A and 10B, using a padding accessory strap 22 and Velcro™ strip 21 to support two pressure pads 26 with their individual Velcro™ backings (patches) 25, shown in greater detail in FIGS. 11C and 11D.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A tension band comprising:
three or more straps configured to form a single band, wherein an end of a first strap is coupled to an end of a second strap by a first tension control system, and the other end of the second strap is coupled to an end of a third strap by a second tension control system; and
a closure system for securing the free ends of the band around a body part of a subject,
wherein when the band is secured around the body part of a subject, each tension control system can be independently operated by the subject to adjust the degree of pressure applied by the band to the body part.

2. The tension band according to claim 1, wherein each tension control system comprises a tension adjustment mechanism operatively associated with a tension member which spans the coupled ends of two straps, wherein the subject uses the tension adjustment mechanism to change the degree of pressure applied by the band to the body part by adjusting the length of the tension member that spans the coupled ends of two straps.

3. The tension band according to claim 2, wherein the tension member is a cable that spans and is affixed proximal to the coupled ends of straps, and the tension adjustment mechanism is affixed proximal to one of the coupled ends of said two straps.

4. (canceled)

5. The tension band according to claim 2, wherein the tension adjustment mechanism of the first tension control system is affixed proximal to the end of the first strap and the tension adjustment mechanism of the second tension control system is affixed proximal to the end of the third strap.

6. The tension band according to claim 3, wherein the ends of the cable are housed within the tension adjustment mechanism.

7. The tension band according to claim 1, wherein the body part is the head, pelvis, or lower back of the subject and each tension control system is positioned substantially on a side of the head, pelvis, or lower back of the subject when the band is secured to said body part.

8. (canceled)

9. (canceled)

10. A method of using of a tension band to apply pressure to a body part of a subject, the tension band comprising:
three or more straps configured to form a single band, wherein an end of a first strap is coupled to an end of a second strap by a first tension control system, and the other end of the second strap is coupled to an end of a third strap by a second tension control system; and
a closure system for securing the free ends of the band around a body part of a subject, wherein when the band is secured around the body part of a subject, each tension control system can be independently operated by the subject to adjust the degree of pressure applied by the band to the body part.

11. The method of using the tension band according to claim 10, wherein each tension control system comprises a tension adjustment mechanism operatively associated with a tension member which spans the coupled ends of two straps, wherein the subject uses the tension adjustment mechanism to change the degree of pressure applied by the band to the body part by adjusting the length of the tension member that spans the coupled ends of two straps.

12. The method of using the tension band according to claim 11, wherein the tension member is a cable that spans and is affixed proximal to the coupled ends of straps, and the tension adjustment mechanism is affixed proximal to one of the coupled ends of said two straps.

13. The method of using the tension band according to claim 11, wherein the tension adjustment mechanism of the first tension control system is affixed proximal to the end of the first strap and the tension adjustment mechanism of the second tension control system is affixed proximal to the end of the third strap.

14. The method of using the tension band according to claim 12, wherein the ends of the cable are housed within the tension adjustment mechanism.

15. The method of using the tension band according to claim 11, wherein each tension adjustment mechanism is mounted on a semi-rigid backing.

16. The method of using the tension band according to claim 10, wherein the body part is the head, pelvis, or lower back of the subject and each tension control system is positioned substantially on a side of the head, pelvis, or lower back of the subject when the band is secured to said body part.

17. A method of making a tension band comprising the steps of:
connecting three or more straps configured to form a single band, wherein an end of a first strap is coupled to an end of a second strap by a first tension control system, and the other end of the second strap is coupled to an end of a third strap by a second tension control system; and
providing a closure system for securing the free ends of the band around a body part of a subject,
wherein when the band is secured around the body part of a subject, each tension control system can be independently operated by the subject to adjust the degree of pressure applied by the band to the body part.

18. The method of making the tension band according to claim 17, wherein each tension control system comprises a tension adjustment mechanism operatively associated with a tension member which spans the coupled ends of two straps, wherein the subject uses the tension adjustment mechanism to change the degree of pressure applied by the band to the body part by adjusting the length of the tension member that spans the coupled ends of two straps.

19. The method of making the tension band according to claim 18, wherein the tension member is a cable that spans and is affixed proximal to the coupled ends of straps, and the tension adjustment mechanism is affixed proximal to one of the coupled ends of said two straps.

20. The method of making the tension band according to claim 18, wherein the tension adjustment mechanism of the first tension control system is affixed proximal to the end of the first strap and the tension adjustment mechanism of the second tension control system is affixed proximal to the end of the third strap.

21. The method of making the tension band according to claim 19, wherein the ends of the cable are housed within the tension adjustment mechanism.

22. The method of making the tension band according to claim 18, wherein each tension adjustment mechanism is mounted on a semi-rigid backing.

23. The method of making the tension band according to claims 17, wherein the body part is the head, pelvis or lower back of the subject and each tension control system is positioned substantially on a side of the head, pelvis, or lower back of the subject when the band is secured to said body part.

24. The tension band according to claim 2, wherein each tension adjustment mechanism is mounted on a semi-rigid backing.
